(19) United States
(12) Patent Application Publication
(10) Pub. No.: US 2025/0268715 A1
(43) Pub. Date: Aug. 28, 2025

(54) MITRALLEAFLET TETHERING

(71) Applicant: PIPELINE MEDICAL TECHNOLOGIES, INC., Newark, DE (US)

(72) Inventors: Steven F. Bolling, Ann Arbor, MI (US); Randall T. Lashinski, Windsor, CA (US)

(73) Assignee: PIPELINE MEDICAL TECHNOLOGIES, INC., Newark, DE (US)

(21) Appl. No.: 19/204,945

(22) Filed: May 12, 2025

Related U.S. Application Data
(63) Continuation of application No. 18/335,589, filed on Jun. 15, 2023, now Pat. No. 12,318,296.

(51) Int. Cl. A61F 2/24; A61B 17/00; A61B 17/04; A61M 25/00; A61M 25/01

(52) U.S. Cl. CPC A61F 2/2466; A61B 17/0401; A61B 17/0469; A61F 2/2457; A61M 25/0054; A61M 25/0147; A61B 2017/00292; A61B 2017/00876; A61B 2017/0409; A61B 2017/0419; A61B 2017/0427; A61B 2017/0441; A61B 2017/048; A61F 2220/0008; A61F 2220/0016; A61F 2230/0091

(57) ABSTRACT

Apparatuses and techniques to access the right ventricle via transfemoral vein threading a catheter or catheters to the apex or bottom of the right ventricle. Piercing through the venous or right side of the heart in the interventricular septal wall to access the left ventricle a catheter can be passed to turn upward pointing to the mitral valve. From this access point in the left ventricle the flail mitral leaflet can be sutured and tethered pulling it back into position and reattached with a grounding anchor in the right ventricle or imbedding the anchor into the septal wall. The interventricular septal wall crossing technique could include the passing of a coaxial catheter through the first access catheter where the first access catheter could act as a guide to direct the internal or second coaxial catheter toward the flail mitral leaflet.

### INCORPORATION BY REFERENCE TO ANY PRIORITY APPLICATIONS

This application is a continuation of U.S. application Ser. No. 18/335,589, filed Jun. 15, 2023, which is a continuation of U.S. application Ser. No. 16/024,439, filed Jun. 29, 2018, which is a continuation of PCT Patent Application No. PCT/US2016/069567, filed Dec. 30, 2016, which claims a priority benefit to U.S. Provisional Application No. 62/383,338, filed Sep. 2, 2016 and U.S. Provisional Application No. 62/273,300, filed Dec. 30, 2015, the entire disclosure of these provisional applications are hereby incorporated by reference herein for all purposes in their entireties and should be considered a part of this specification.

### BACKGROUND

### Field

The disclosure relates generally to cardiac treatment devices and techniques, and in particular, to methods and devices for mitral valve repair.

### Description of the Related Art

The heart includes four heart valves, which allow blood to pass through the four chambers of the heart in one direction. The four valves are the tricuspid, mitral, pulmonary and aortic valves. The four chambers are the right and left atria (upper chambers) and right and left ventricle (lower chambers).

The mitral valve is formed by two leaflets, which are known as the anterior leaflet and the posterior leaflet, which open and close in response to pressure placed on the leaflets by the pumping of the heart. There are several problems that can develop or occur with respect to the mitral valve. Such problems include mitral valve regurgitation (MR), in which the mitral valve leaflets do not close properly, which can cause leakage of the mitral valve. Severe mitral regurgitation can adversely affect cardiac function and compromise a patient's quality of life and life-span. There are several techniques directed to correcting mitral valve regurgitation, which include valve replacement, chordae tendinea shortening or replacement and mitral annular repair also known as annuloplasty.

Current techniques to correct mitral regurgitation include repairing the mitral valve via open heart surgery while a patient's heart is stopped and the patient is on cardiopulmonary bypass. Such techniques are highly invasive that have inherent risks. It would be desirable to provide a less invasive procedure for repairing a mitral valve.

### SUMMARY

One embodiment disclosed herein includes a method of repairing a mitral valve of a patient's heart that comprises accessing a right ventricle of the patient's heart with a catheter extending through a venous or right side of the heart to access the left ventricle and and with the catheter securing a mitral valve leaflet.

Another embodiment disclosed herein is a chordae replacement system that can include a catheter and a chordae replacement implant. The catheter can have an elongate, flexible tubular body with a proximal end and a distal end. The catheter can be configured for transvascular access into the right ventricle, through the intraventricular septum and into the left ventricle. The chordae replacement implant can be deployably carried by the catheter. The chordae replacement implant can comprise an elongate body having a proximal end with a proximal tissue anchor and a distal end with a mitral valve leaflet attachment anchor.

Another embodiment disclosed herein is method of repairing a mitral valve, the method comprising with a catheter transvascularly accessing the right ventricle and extending the catheter through the intraventricular septum and into the left ventricle and deploying a chordae replacement implant with the catheter.

### BRIEF DESCRIPTION OF THE DRAWINGS

FIG. 1AA illustrates the normal mitral leaflet connections in the left ventricle include chordal attachments from the free margin of the mitral leaflet to the papillary muscles.

FIG. 1A illustrates a ruptured chordal attachment.

FIG. 1 illustrates a technique to access the right ventricle via trans-femoral vein threading a catheter or catheters to the apex or bottom of the right ventricle.

FIG. 2 illustrates a catheter piercing through the venous or right side of the heart in the interventricular septal wall to access the left ventricle.

FIG. 3 illustrates first and second catheters that could be steered to position the distal tip to capture the margin of the mitral leaflet.

FIG. 4 illustrates magnets that can be used to position the tips of two catheters relative to one another.

FIG. 4A illustrates passing a suture loop through the mitral leaflet and tethered back through a lower catheter and attached to the anchor at the apex or intraventricular septal wall.

FIG. 5 illustrates a grounding plug.

FIG. 5A illustrates an internal anchor within the tissue wall separating the left and right ventricle or interventricular septal wall tissue.

FIG. 5AA illustrates a septal anchor

FIG. 5B illustrate embodiments of an internal anchor and apex anchor.

FIG. 6 illustrates a grounding anchor positioned within the heart.

FIG. 7 illustrates a coiled anchor.

FIG. 8 illustrates access from the jugular vein would also provide access into the vena cava and right ventricle and or access into the left atrium via trans-septal puncture.

### DETAILED DESCRIPTION OF THE PREFERRED EMBODIMENTS

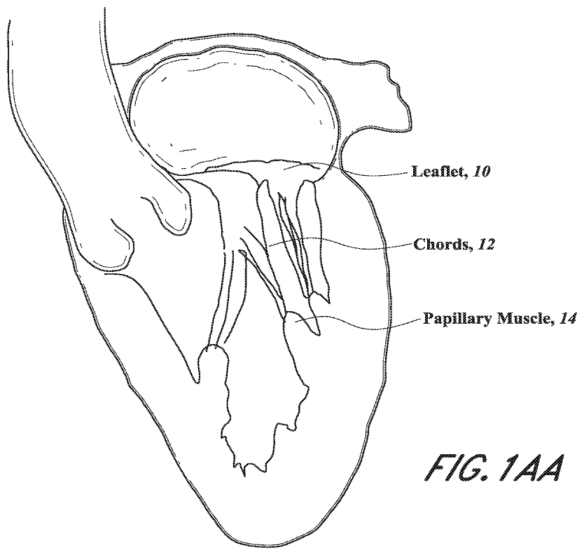

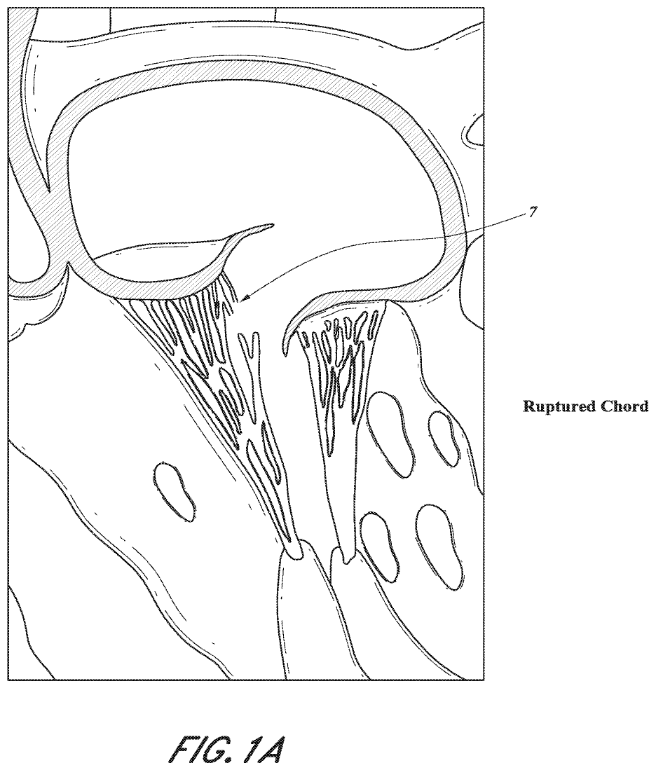

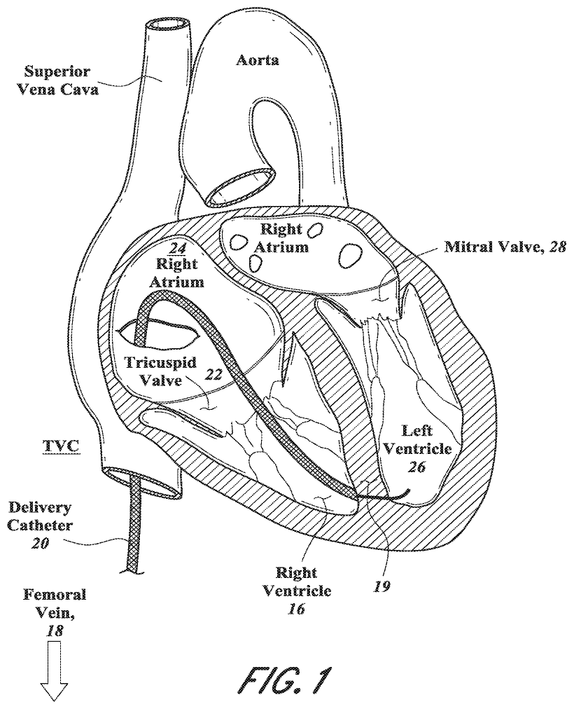

Normal mitral leaflet 10 connections in the left ventricle include chordal attachments 12 from the free margin of the mitral leaflet 10 to the papillary muscles 14, which are shown in FIG. 1AA.

The repair and reconnection of a flail leaflet (a ruptured chord 7 being shown in FIG. 1A) surgically can be completed with a suture by reattaching the leaflet to a papillary muscle. Another technique would be a trans-apical reconnection of the flail leaflet similar to a technology developed by a company named NeoChord.

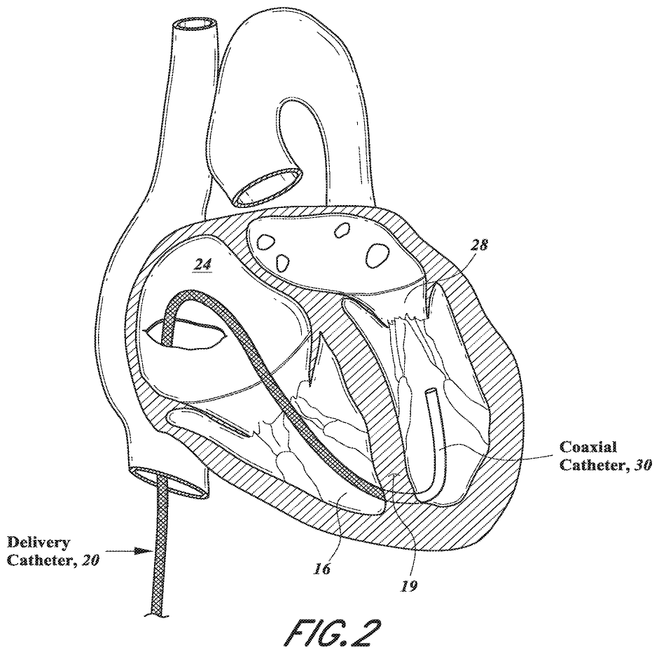

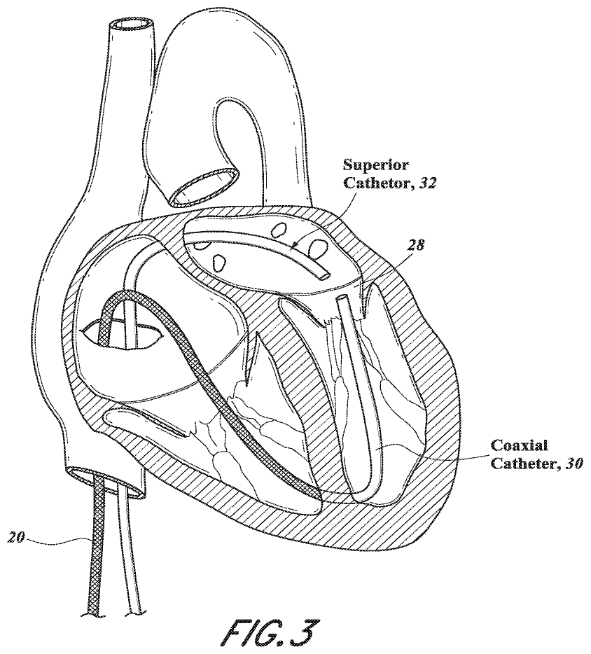

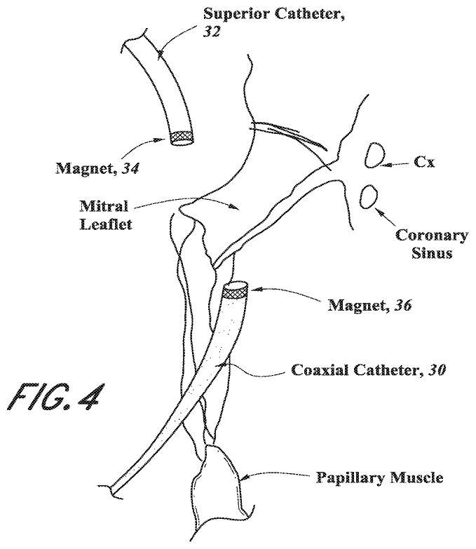

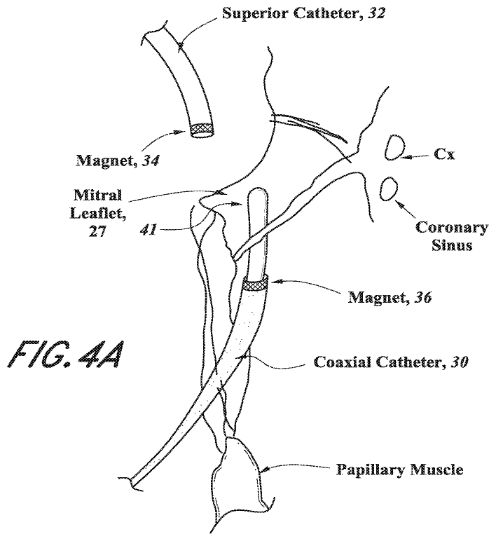

A different technique would be to access the right ventricle 16 via trans-femoral vein 18 threading a catheter 20 or catheters to the apex or bottom of the right ventricle 16 as shown in FIG. 1. The entry would start in the femoral vein 18 in the groin proceeding up through the inferior vena cava into the right atrium 24 through the tricuspid valve 22 to the bottom of the right ventricle 16. Piercing through the venous or right side of the heart in the interventricular septal wall 19 to access the left ventricle 26 a catheter 20 can be passed to turn upward pointing to the mitral valve 28 as shown in FIG. 2. From this access point in the left ventricle 26 the flail mitral leaflet can be sutured and tethered pulling it back into position and reattached with a grounding anchor in the right ventricle 16 or imbedding the anchor into the septal wall. The interventricular septal wall crossing technique could include the passing of a coaxial catheter 30 through the first access catheter 20 where the first access catheter 20 could act as a guide to direct the internal or second coaxial catheter 30 toward the flail mitral leaflet. Both first and second catheters 20, 30 could be steerable to position the distal tip direction to capture the margin of the mitral leaflet as shown in FIG. 3. A piercing needle could be passed to thread a suture through the mitral leaflet for reattachment or the leaflet to the lower chamber of the heart, into the septal wall or transvers the septal wall and anchor in the right ventricle. Threaded a tether through the mitral leaflet and back through the second internal catheter 30 and attached to the grounding anchor, the leaflet would be pulled into proper position replicating a chordal attachment that may have failed or broken. The attachment of the new suture to the grounding anchor could be achieved through a knot, sliding one-way stopper or other means to join the anchor and suture together. A single line attachment or a plurality of lines would allow the load to be shared or pulled in different force vectors moving the grounding point of the mitral leaflet in different directions. As shown in FIG. 3, a secondary atrial access could be achieved through the venous system superiorly to the mitral valve via trans-septal puncture to pass an additional catheter 32 into the left atrium for positioning above the flail leaflet. Achieving a second securement of the leaflet from above along with below would allow for positive positioning and suture attachment within the leaflet margin as viewed under echo and fluoroscopy. At the tip of each catheter could be a magnet 36, 34 to position the tips of each catheter 30, 32 relative to one another as shown in FIG. 4.

The magnet 36, 34 could have a through-hole or central lumen to pass wires, suture 43 or other items longitudinally from one tip to another. A suture loop 41 would be passed through the mitral leaflet 27 and tethered back through the lower catheter 30 and attached to the anchor at the apex or intraventricular septal wall as shown in FIG. 4A.

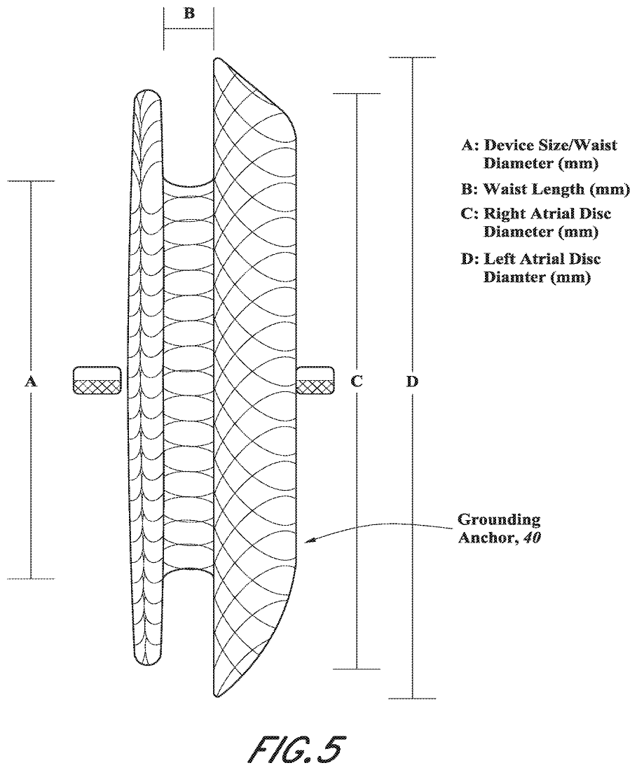

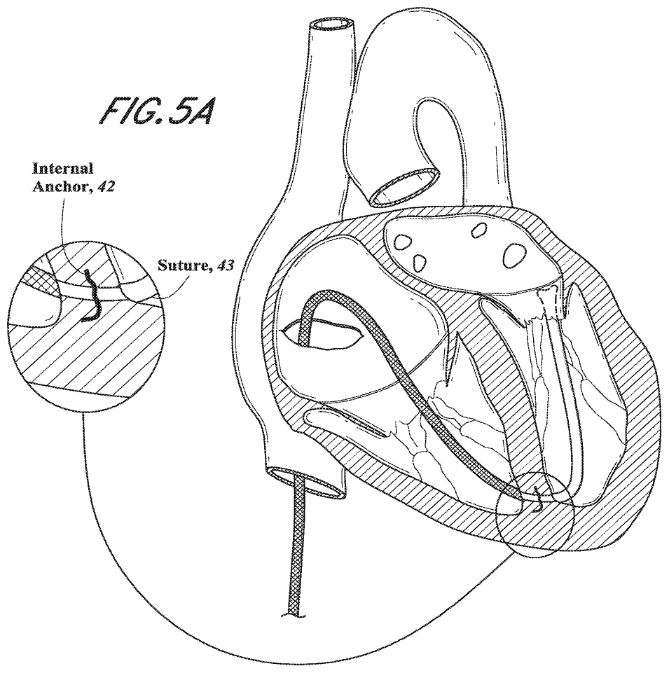

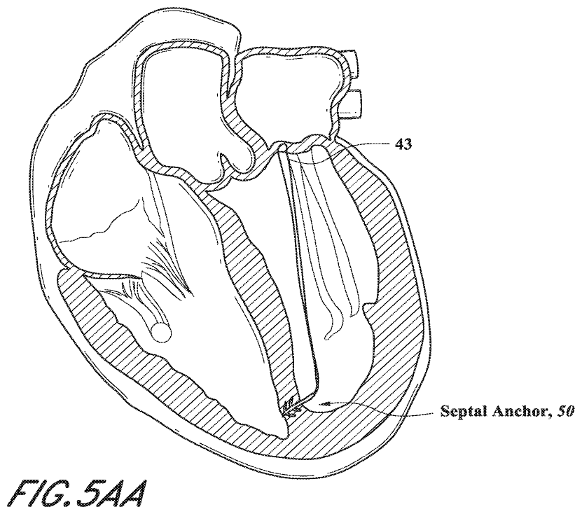

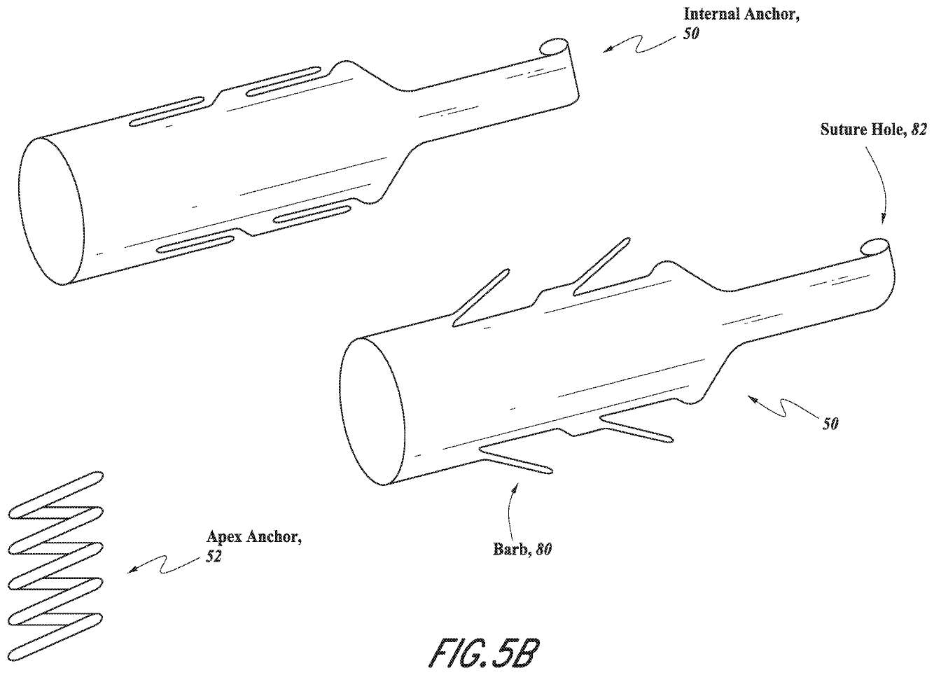

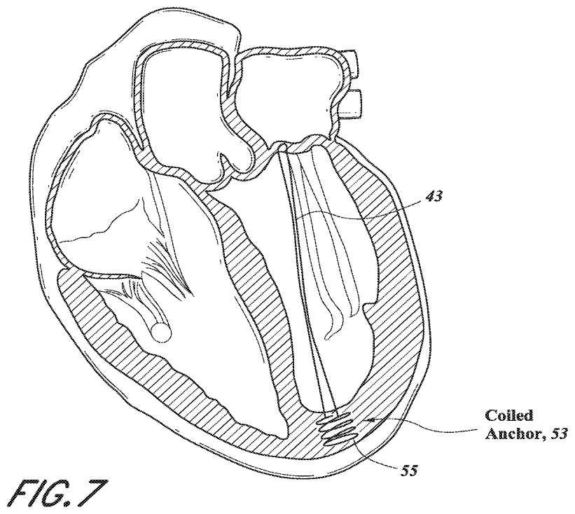

The grounding plug or anchor 40 could be similar to an Amplatz device used for closing an ASD or another device to distribute forces to a larger area distributing the load throughout a larger surface area in the right ventricle or within the interventricular septal wall as shown in FIG. 5 Another means to secure the sutures within the right ventricle would be attach them to a pledget 73 or other pad to spread the load within the right ventricle. One alternative technique would be to imbed an internal anchor 42 within the tissue wall separating the left and right ventricle or interventricular septal wall tissue as shown in FIG. 5A. This internal anchors 50 of FIGS. 5B and 5AA could be delivered from above, or from the left atrium, through the septal access and passing through the mitral leaflet to connect the mitral leaflet to the suture 43 and into the septal wall between the right and left ventricle securing it to an internal structure such as an anchor to resist movement during the tensioning of the suture line. As shown in FIG. 5A, the internal anchor can include barbs 80 and a suture hold 82. It may also be advantageous to extend a section of the anchor into the left atrium away from the septal wall to position the tangent point directly below the attachment point of the mitral leaflet. This would provide a direct line to the attachment points above and below without a torque or moment about the entry to the septal wall and not interfere with any other chordal structures or papillary muscles. A strain relief at the anchor exit may also prohibit fretting of the suture line as its cyclical loading may be an area of stress concentration. Also a coiled anchor 52 (see FIG. 5B) could be delivered from above with a trans-septal access through the mitral valve and into the apex of the heart or into the myocardial tissue as shown in FIG. 7. The coil 55 would allow a contact point connected to the suture line which is farther connected to the mitral leaflet. A plurality of connection points could also be added for additional support or to tether additional ruptured chords. A secondary adjustment could also occur by re-tethering the connection lines by winding, re-knotting or pulling the suture lines post implant procedure.

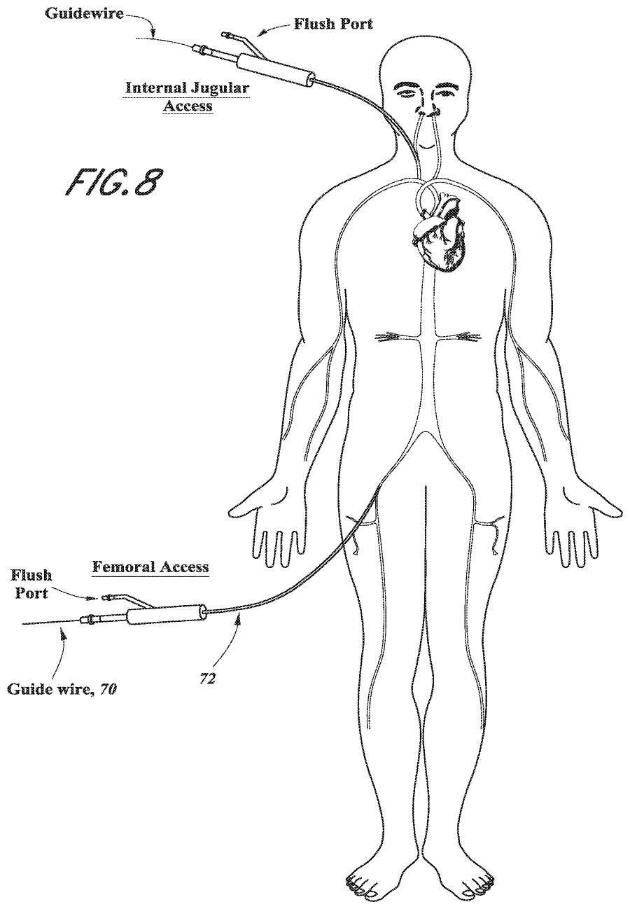

Access into the femoral vein could occur with a guidewire 70 measuring about 0.035 inches in diameter and about 180 centimeters in length. An introducer sheath could follow to provide a conduit to pass additional catheters in and out of the femoral access site as shown in FIG. 8. The catheter 72 could measuring about 10 to 24 French in diameter the introducer could be advanced into the femoral vein with a dilator to guide the tip without vessel trauma. The length of the catheter 72 could be about 100 centimeters in length. Advancing the device delivery catheter through this introducer sheath over the guidewire 72 could provide a radiopaque means for tracking the guidewire, introducer sheath and delivery catheter via live x-ray or fluoroscopy. Passed into the inferior vena cava and turning into the right atrium through the tricuspid valve, the catheter can follow the guidewire or be actively shaped or bent through a deflectable catheter at the handle via pull-wire or shaping system. Contrast dye injected into the heart can provide a road map to structural items within the heart. Aiming or steering the catheter and guidewire to the apex of the right ventricle and passing a needle or piercing tool to pass from the right ventricle to the left ventricle will provide access from the femoral vein to the left ventricle accessing the mitral valve.

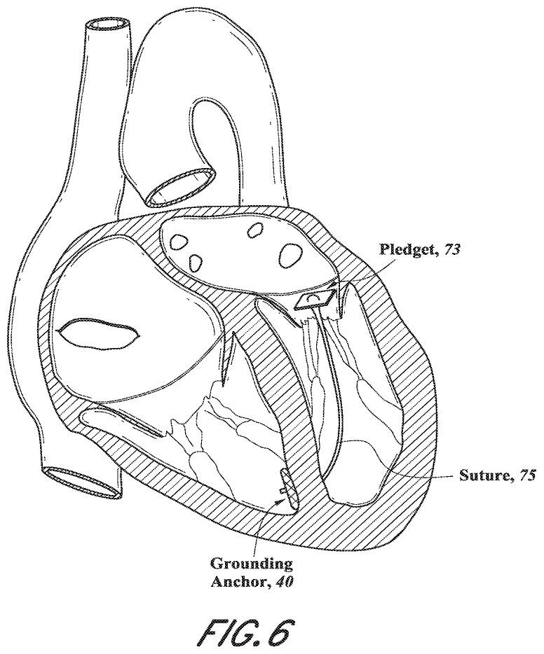

An access pathway to the left atrium through a transseptal puncture can be completed by also via the femoral artery at the groin to advance a guidewire and catheter system in a similar manor as described above. This would allow for an above and below intimate contact of the mitral valve leaflets to secure and suture them back into proper positioning. The above-catheter from the left atrium and below-catheter from the left ventricle, via right ventricle, can locate and hold the position of the flail leaflet for suture piercing and tethering back into its proper position to coapt with the adjacent leaflet eliminating the mitral regurgitated blood flow. Piercing needles and strain relieving pledgets 75 could be used to pass suture 75 and distribute the local forces at the leaflet attachment site as shown in FIG. 6. Single or multiple passes through the leaflet will provide a duplication of the normal chorde providing normal leaflet motion. The suture material can be #4 or #5 PTFE, Silk or other common materials used in normal valve repair. The position of the suture would allow for normal left ventricle and mitral valve motion and freedom as the suture would pass in between the papillary muscles and connect to the flail leaflet at one end and into the right ventricle at the other end held by a strain relief in the right ventricle. Access from the jugular vein would also provide access into the vena cava and right ventricle and or access into the left atrium via trans-septal puncture as shown in FIG. 8. This jugular access would eliminate the first 180 degree turn up the femoral vein and into the right ventricle but is not a conventional access for most interventional cardiologist.

Catheters would be constructed of common polymers including nylon, Teflon, urethanes, and other commonly used materials having a proximal and distal end with a guidewire port through s The catheter curves needed would be pre-set, fixed or actively curved through differential forces transmitted via pull wires or tubes to bias one direction or another providing a column compression on one side of the catheter relative to the other. Column and tubular strength could be provided by imbedded coiled wires, braided with ribbon or round wire, laser cut tubes or skeletal structures to form a defined structure and or curve needed to gain access. Variable durometers, construction techniques are well known in the industry to allow for specific pushability, stiffness and curves needed to deliver. Coatings and surface treatments both internally and externally could aid in relative movement between vessel walls and between wires and other catheters. The tensioning means could be provided by a pull-wire extending from the distal end of the catheter to the handle of the proximal section. This pull wire could be activated by rotational screws translated into longitudinal forces pulling a connection to the distal end of the catheter. The overall length of the femoral catheter access would be about 100 cm in length and have a through lumen to accept a guidewire for positioning within the bodies vasculature. The overall length of the internal jugular catheter would be about 60 cm in length. Both catheters would be about 6-20 French in diameter with at least one lumen from the proximal distal end of the delivery system.

Access from the femoral vein will allow for catheterization through the tricuspid valve and into the right ventricle. At the apex of the right ventricle an access will be attained by advancing a needle or catheter in through the septal wall gaining access to the left ventricle. Use of a needle, ultrasonic or coring tool to pass a guidewire from right ventricle to left ventricle is the pathway and access route to repair the mitral valve. Once a needle and or guidewire can be advanced additional tools such as catheters can be utilized to repair the mitral valve. The septal wall can be over 1 centimeter in thickness so maintaining an access port may be achieved by a balloon dilatation, guide catheter or access conduit to pass tools and catheters through during the repair. A steerable sheath, catheter or conduit may allow an easier access direction to the specific area of the mitral valve for repair. Adjustments made rotationally and or angularly can be fixed or locked into position once optimal positioning is obtained. This can be achieved by a pre-shaped curve configuration whereas the catheter is curved down through the tricuspid valve and across the ventricular septal wall then pointing upward toward the mitral valve. This shape can be fixed or variable based upon patient needs and anatomy. Guidewires measuring about 0.035 inches in diameter and about 180 to 300 centimeters in length will allow for catheters to be advanced over and allow exchange of additional tools to be interchanged. Expandable dilators can be used expand areas where tight access is required or larger bore catheters are required. Catheter sizing may start from about 6 French to about 24 French in diameter and range from lengths including 90 centimeters to 160 centimeters. Construction of these catheters can be of normal polymers including nylon, polyurethane, polyethylene or other similar polymers. Braids, coils or laser cut tubes can be used within the catheter construction to better support inner diameters, shapes or curves required. These materials can also include stainless steel, Nitinol, Platinum or MP35N metallic suitable for catheter construction.

Nesting multiple catheters inside one another will provide for additional curves, movement, and translational freedoms. In one embodiment a larger catheter (24 French inner diameter) to access the apex of the right ventricle could be used to position a stable base from which to advance an inner catheter (18 French inner diameter) through the ventricular septal wall and a third catheter could be advanced through this catheter measuring about 14 French inner diameter to advance into the left ventricle directing toward the mitral valve. These catheters would allow for multiple adjustments and angles for various anatomies. The ability to translate, rotate and lock position of each of these catheters together or independently will provide a stable platform to deliver repair tools to the valve. Locking means for each of these catheters nested inside one another can be achieved by an expansion via diameter change using a hydraulic pressure, a mechanical expansion via rotational means creating an eccentric lock or a longitudinal pull to create a differential diameter between the catheters. This push-pull translation could force the catheter to accordion creating a larger bump within one catheter.

Additionally, push pull wires could force the catheters into predetermined shapes and curves in single or multiple plains. By laser cutting a specific pattern into the catheter inner frame a shape can be forced by a pull wire reducing one side of the catheter length while collapsing the round column shape of the catheter creating a shape as determined by the laser cut element internal to the catheter. As an example, a slot could be cut into one side of the tube and a tension wire attached at the distal end of the tube. As tension is applied to the wire, a collapsing of the slotted side of the tube would result in curve or bias to the tubular element. These slots could also be complex shapes to lock the rotational angle into a pre-determined shape. This complex shape could be a chevron, angled cut, radiused shape or another detailed pattern to stop the collapsing of the tube at a predetermined radius. This pattern could also be rotated about the tube to create three-dimensional shapes and curves out of a single plane.

This patterning would be laser cut into the inner tube of the catheter and be constructed from a metallic or polymer and embedded into the wall of the catheter wall.

The first angular curve would be about 180 degrees changing the direction of the catheter from the femoral access through the tricuspid valve and directing toward the apex of the right ventricle. The second curve in this catheter would be about a 90 degree turn toward the ventricular septal wall making a “Shepard's Crook” shape. This 90 degree direction could be also attained with a second inner catheter passed through the first larger diameter catheter to direct the access through the ventricular septal wall. This would require a 90 degree curve to redirect the tip toward the septal wall. Once a penetration of the septal wall is achieved another 90 degree curve would be required to direct the catheter toward the mitral valve. Between these two 90 degree curves and separation of about 1 to 2 centimeters is required to traverse the septal wall tissue. This straight section could be preshaped into the curve configuration and be actuated with a single pull wire or multiple pull wires. The preferred embodiment would utilize the first catheter to attain the 90 degree curve toward the ventricular septal wall.

The next inner catheter directed toward the mitral valve could be advanced toward the valve leaflets in the left ventricle. Directed and placed below the leaflet the catheter tip could locate the free margin of the mitral valve leaflet to secure a tether for a ruptured chord or flail leaflet repair. Single or multiple chords can emanate from a single access point or from separate locations along the valve leaflet. From above through a transseptal access a second catheter could located the top side of the leaflet along the same free margin of the leaflet. Locating these two catheters coaxially could be achieved through a magnetic tip that is built into the catheter or advanced through each central lumen of the catheters.

Locating these two catheters above and below the leaflet pinching one another with the leaflet sandwiched in between would allow for an access through the leaflet for chordal repair or tethering to secure through the lower access point originating from the right ventricle. The chordal repair could be a PTFE suture or another material suitable for permanent implantation. With the lower access point extending into the right ventricle an anchor could be located completely in the right ventricle or within the ventricular septal wall exposing only the replacement suture material in the left ventricle. Anchor designs can be similar to a barbed anchor with a single or plurality of barbs to engage the tissue, a plug to hold from the right ventricle side of the septal wall or a screw means to engage the tissue in the right or left ventricle. The attachment of the tissue anchor to the chordal leaflet attachment can be adjusted while monitoring the tension of the chord or the echo results live during or after the implantation of the chords and anchor system.

1. A chordae replacement system, comprising:
a steerable catheter having an elongate, flexible tubular body with a proximal end and a distal end, the catheter configured for transvascular access into the right ventricle, through the intraventricular septum and into the left ventricle, the steerable catheter including a pull-wire extending from a handle at a proximal section of the steerable catheter to a distal end of the steerable catheter, the pull-wire configured to actively bend the steerable catheter to position a distal tip of the steerable catheter adjacent a mitral leaflet;
a chordae replacement implant deployably carried by the catheter, the chordae replacement implant comprising an elongate body having a proximal end with a proximal tissue anchor and a distal end with a mitral valve leaflet attachment anchor, wherein the elongate body is a suture of a material suitable for permanent implantation; and
a piercing needle configured to be passed within the steerable catheter and to thread the suture through the mitral leaflet at a leaflet attachment site.

2. The chordae replacement system of claim 1, wherein the proximal tissue anchor comprises a helical tissue anchor for engaging tissue at the apex of the heart.

3. The chordae replacement system of claim 1, wherein the proximal tissue anchor comprises an anchor for engaging the right ventricular surface of a trans septal crossing.

4. The chordae replacement system of claim 1, wherein the chordae replacement implant is carried within the tubular body.

5. The chordae replacement system of claim 1, wherein the steerable catheter includes slots configured to collapse as tension is applied to the pull-wire to curve the steerable catheter through a predetermined curve.

6. The chordae replacement system of claim 1, wherein the steerable catheter is a first catheter, the catheter-based system further comprising a second coaxial catheter configured to guide the first catheter through the septal wall and toward the mitral leaflet.

7. The chordae replacement system of claim 6, wherein the first and second catheters are both steerable catheters.
